(19) United States
(12) Patent Application Publication
(10) Pub. No.: US 2019/0008735 A1
(43) Pub. Date: Jan. 10, 2019

(54) METHOD OF INCREASING THE WASH RESISTANCE OF ARTIFICIALLY COLORED HAIR

(71) Applicant: Henkel AG & Co. KGaA, Duesseldorf (DE)

(72) Inventors: Erik Schulze zur Wiesche, Hamburg (DE); Rene Krohn, Norderstedt (DE)

(73) Assignee: Henkel AG & Co. KGaA, Duesseldorf (DE)

(21) Appl. No.: 16/131,977

(22) Filed: Sep. 14, 2018

Related U.S. Application Data
(62) Division of application No. 15/444,095, filed on Feb. 27, 2017.
Application No. 15/444,095 is a continuation of application No. PCT/EP2015/069403, filed on Aug. 25, 2015.

(30) Foreign Application Priority Data
Aug. 29, 2014 (DE) 10 2014 217 310.8

(51) Int. Cl. A61K 8/365; A61K 8/19; A61Q 5/00; A61Q 5/02

(52) U.S. Cl. CPC A61K 8/365; A61K 8/19; A61Q 5/02; A61Q 5/004

(57) ABSTRACT

A method of reducing and/or preventing the bleaching and/or fading of artificially produced hair colors, includes the following steps: i. applying a cosmetic composition to the preferably wet, colored hair, ii. allowing the composition to act for a period of at least 5 s, iii. optionally: rinsing the composition out with water. The cosmetic composition, based on the weight thereof, includes a) 0.05 to 0.5 mol/kg of at least one divalent salt of a monobasic organic acid or b) 0.01 to 0.5 mol/kg of at least one monobasic organic acid (b1) and 0.05 to 0.5 mol/kg of at least one divalent inorganic salt (b2), and the cosmetic composition has a pH in the range from 4.0 to 4.8.

### FIELD OF THE INVENTION

The invention lies in the field of cosmetics and relates to a method for reducing and/or preventing the bleaching and/or fading of artificially produced hair colors, comprising the step of applying a specific cosmetic composition to the hair.

### BACKGROUND OF THE INVENTION

The invention also relates to a cosmetic cleaning and nourishing composition for reducing and/or preventing the bleaching and/or fading of artificially produced hair colors, which, in addition to an anionic and/or a cationic surfactant, also includes at least one divalent salt of a monobasic organic acid and/or at least one monobasic organic acid and at least one divalent inorganic salt.

Products for changing the natural hair color play a prominent role in the field of hair cosmetics. A distinction is made between permanent, semi-permanent or temporary coloring systems based on chemical and/or natural dyes. The hair colors artificially produced by permanent, semi-permanent or temporary coloring systems, however, have the disadvantage that they can change undesirably, for example during or after cleaning of the hair.

The term “undesirable change” is understood to mean the fading or bleaching and also loss of color brilliance of the chroma of the hair achieved by the dyeing. Ambient influences and/or the effects of the sun can intensify these changes further still.

There is thus a need for cosmetic methods with which artificially produced hair dyes can be better stabilized.

Hair treatment agents for protecting artificially produced hair colors or methods for stabilizing artificially produced hair colors are known in principle. Document EP 1676604 Al describes a method for improving the chroma of hair in which the hair is first washed with a shampoo including at least one water-soluble salt, preferably sodium sulfate, in addition to an anionic surfactant and a specific silicone. In a second step, the hair is treated with a conditioning agent comprising a higher alcohol and a cationic surfactant in a specific ratio by weight, and the conditioning agent is then washed out.

However, consumers desire quicker methods with which hair colors can be protected preferably in one treatment step before any change occurs.

It is therefore desirable to develop an easily and quickly performed cosmetic treatment method with which the adhesion of dyes to the hair fibres is strengthened, thus resulting in improved fastness of the artificially produced hair color. Ideally, mild cosmetic compositions which provide the treated hair, in addition to the color protection, with improved properties in terms of appearance and feel and which do not stress the hair should be used in the method.

### BRIEF SUMMARY OF THE INVENTION

A first subject of the invention is therefore a method for reducing and/or preventing the bleaching and/or fading of artificially produced hair colors, said method comprising the following steps:
a) applying a cosmetic composition to the colored hair, which is preferably wet,
b) allowing the composition to act for a period of at least 5 seconds,
c) optionally: rinsing the composition out using water, characterized
   in that the cosmetic composition includes—in relation to its weight—
      a) 0.05 to 0.5 mol/kg of at least one divalent salt of a monobasic organic acid or
      b) 0.01 to 0.5 mol/kg of at least one monobasic organic acid (b1) and 0.05 to 0.5 mol/kg of at least one divalent inorganic salt (b2), and
   in that the cosmetic composition has a pH value in the range of from 4.0 to 4.8.

A cosmetic cleaning or nourishing composition for reducing and/or preventing the bleaching and/or fading of artificially produced hair colors, including—in relation to its weight—
a)
   0.05 to 0.5 mol/kg of at least one divalent salt of a monobasic organic acid or
   0.01 to 0.5 mol/kg of at least one monobasic organic acid (b1) and 0.05 to 0.5 mol/kg of at least one divalent inorganic salt (b2)
b) at least one surfactant, selected from
   anionic surfactants of the group formed of alkyl(ether)sulfates, sulfosuccinates, ether carboxylic acids, acylglutamate and/or (acyl)isethionates, each with 8 to 24 C atoms in the acyl group, and mixtures of these surfactants, and/or
   cationic surfactants of the group formed of quaternary ammonium compounds, esterquats and/or amidoamines,
characterized in that the cleaning or nourishing composition has a pH value ranging from 4.2 to 4.7.

### BRIEF DESCRIPTION OF THE DRAWINGS

The present invention will hereinafter be described in conjunction with the following drawing figures, wherein like numerals denote like elements, and

FIG. 1 shows the delta-E values.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has been found that a hair treatment method with use of an acidic cosmetic composition including a specific salt or a specific acid-salt mixture is outstandingly suitable for this purpose.

The term “suitable cosmetic compositions” preferably means hair cleaning agents such as shampoos, haircare agents such as hair masks, conditioners or haircare sprays, and also hairstyling agents such as hair gels, hairsprays or hair waxes.

In a first preferred embodiment, the method according to the invention comprises the following steps
a) applying a shampoo or a hair conditioner to the wet, dyed hair,
b) allowing the shampoo or the hair conditioner to act for a period of from 5 seconds to 3 minutes,
c) rinsing the shampoo or the hair conditioner out using water,
   characterized
   in that the shampoo or the hair conditioner includes—in relation to its weight—
      a) 0.05 to 0.5 mol/kg of at least one divalent salt of a monobasic organic acid or
      b) 0.01 to 0.5 mol/kg of at least one monobasic organic acid (b1) and 0.05 to 0.5 mol/kg of at least one divalent inorganic salt (b2), and
         in that the shampoo or the hair conditioner has a pH value in the range of from 4.0 to 4.8.

In a second preferred embodiment, the method according to the invention comprises the step of applying a hair(care) spray, hair gel or hair wax to the dry, dyed hair, wherein
the hair(care) spray, hair gel or hair wax includes—in relation to its weight—
   a) 0.05 to 0.5 mol/kg of at least one divalent salt of a monobasic organic acid or
   b) 0.01 to 0.5 mol/kg of at least one monobasic organic acid (b1) and 0.05 to 0.5 mol/kg of at least one divalent inorganic salt (b2), and
has a pH value in the range of from 4.0 to 4.8.

Suitable divalent salts (component a)) which can be used in the cosmetic compositions of the method according to the invention are preferably selected from alkaline earth metal salts and/or zinc salts of the monobasic organic acids from the group formed of formic acid, acetic acid, glycolic acid, gluconic acid, lactic acid, mandelic acid and/or glyoxylic acid.

Particularly preferred salts a) are calcium, magnesium and/or zinc salts of the aforementioned acids. Calcium salts, such as calcium gluconate and calcium lactate, are very particularly preferred. Calcium lactate is preferred in particular.

Suitable components b) which can be used in the cosmetic compositions of the method according to the invention are preferably selected from monobasic organic acids from the group formed of formic acid, acetic acid, glycolic acid, gluconic acid, lactic acid, mandelic acid and/or glyoxylic acid (b1) and also from divalent inorganic salts selected from the group formed of alkaline earth metal halides, alkaline earth metal hydroxides, alkaline earth metal sulfates, alkaline earth metal oxides, zinc halides, zinc hydroxides, zinc sulfates and/or zinc oxides (b2).

Particularly suitable acids (b1) are lactic acid and gluconic acid. Lactic acid is suitable in particular.

Particularly suitable salts (b2) are calcium, magnesium and/or zinc halides and also calcium, magnesium and/or zinc hydroxides. Calcium, magnesium and/or zinc chloride and also calcium, magnesium and/or zinc hydroxide are very particularly preferred. Calcium chloride and calcium hydroxide are preferred in particular.

In a further preferred embodiment of the method according to the invention, the cosmetic composition therefore includes
as component a), preferably salts formed from alkaline earth metal salts and/or zinc salts with formic acid, acetic acid, glycolic acid, gluconic acid, lactic acid, mandelic acid and/or glyoxalic acid, or
as component (b1), preferably formic acid, acetic acid, glycolic acid, gluconic acid, lactic acid, mandelic acid and/or glyoxalic acid, and
as component (b2), preferably an alkaline earth metal halide, alkaline earth metal hydroxide, alkaline earth metal sulfate, alkaline earth metal oxide, zinc halide, zinc hydroxide, zinc sulfate and/or zinc oxide.

In a very particularly preferred embodiment of the method according to the invention, the cosmetic composition therefore includes
as component a), preferably calcium lactate, or
as component (b1), preferably lactic acid, and, as component (b2), preferably calcium chloride and/or calcium hydroxide.

The cosmetic compositions used in the method according to the invention include—in relation to their weight-preferably
0.04 to 0.40 mol/kg, more preferably 0.06 to 0.30 mol/kg, and in particular 0.07 to 0.20 mol/kg of the salt a), or
0.02 to 0.40 mol/kg, more preferably 0.03 to 0.30 mol/kg, and in particular 0.05 to 0.20 mol/kg of the monobasic organic acid (1)1), and
0.05 to 0.40 mol/kg, more preferably 0.06 to 0.30 mol/kg, and in particular 0.07 to 0.20 mol/kg of the divalent inorganic salt (b2).

In a third preferred embodiment of the method according to the invention, the cosmetic composition therefore includes—In relation to its weight—
0.04 to 0.40 mol/kg, more preferably 0.06 to 0.30 mol/kg, and in particular 0.07 to 0.20 mol/kg of the salt a), or
0.02 to 0.40 mol/kg, more preferably 0.03 to 0.30 mol/kg, and in particular 0.05 to 0.20 mol/kg of the monobasic organic acid (1)1), and
0.05 to 0.40 mol/kg, more preferably 0.06 to 0.30 mol/kg, and in particular 0.07 to 0.20 mol/kg of the divalent inorganic salt (b2).

It has been found that the method according to the invention is particularly effective and that the color change of the artificially produced hair color is particularly small after a number of cleaning processes if the cosmetic compositions include calcium lactate or a mixture of lactic acid and calcium chloride and/or calcium hydroxide and have a pH value ranging from 4.1 to 4.75, preferably from 4.2 to 4.7, and in particular from 4.3 to 4.6.

In a fourth preferred embodiment of the method according to the invention, the cosmetic composition therefore preferably has a pH value ranging from 4.1 to 4.75, more preferably from 4.2 to 4.7, and in particular from 4.3 to 4.6.

The method according to the invention can be applied primarily to hair that has been colored using permanent, semi-permanent, or temporary hair dyes. Temporary hair dyes, however, are intended to wash out/fade over time, and therefore the method according to the invention is particularly suitable for application to hair that has been colored using permanent or oxidative hair dyes.

In a further preferred embodiment, the method according to the invention is therefore applied to hair that has been colored using oxidative hair dyes.

A second subject of the invention is a cosmetic cleaning or nourishing composition for reducing and/or preventing the bleaching and/or fading of artificially produced hair colors, which composition includes—in relation to its weight—
a)
   i) 0.05 to 0.5 mol/kg of at least one divalent salt of a monobasic organic acid or
   ii) 0.01 to 0.5 mol/kg of at least one monobasic organic acid (b1) and 0.05 to 0.5 mol/kg of at least one divalent inorganic salt (b2)
b) at least one surfactant, selected from
   anionic surfactants of the group formed of alkyl(ether)sulfates, sulfosuccinates, ether carboxylic acids, acylglutamate and/or (acyl)isethionates, each with 8 to 24 C atoms in the acyl group, and mixtures of these surfactants, and/or
   cationic surfactants of the group formed of quaternary ammonium compounds, esterquats and/or amidoamines,
      and has a pH value ranging from 4.2 to 4.7.

Cosmetic cleaning and nourishing compositions are preferably understood to be hair shampoos, hair rinses and/or hair masks which, in addition to the aforementioned ingredients, also can include further ingredients conventional in the corresponding products.

Further conventional ingredients and examples of standard formulations of the aforementioned hair treatment agents can be found for example in the monograph Karlheinz Schrader: Grundlagen and Rezepturen der Kosmetika (Basic Principles and Formulations of Cosmetics), 2nd edition (1989), Hüthig Buch publishers, Heidelberg, pages 676-848.

Hair colors can generally change most of all during, or after cleaning of the hair. The contact of the hair with water and surfactants, but also the massaging in of shampoo, towel-drying once the shampoo has been rinsed out, or blow-drying during the subsequent drying process can impair the adhesion of the hair color and can lead to an undesirable change in color and/or to less brilliance of the artificially produced hair color. It is therefore particularly desirable for the process of cleaning dyed hair to be practicable in a particularly gentle manner.

It has been found that the process of cleaning dyed hair is particularly gentle if the cleaning and nourishing composition forming the second subject of the invention is formulated as a hair shampoo or as a conditioner which is applied directly after cleaning the hair.

In a first preferred embodiment of the second subject of the invention, the cleaning and nourishing composition according to the invention is therefore formulated as a hair shampoo and preferably includes at least one anionic surfactant in a preferred proportion by weight of from 0.5 to 20% by weight, more preferably from 1 to 15, and in particular from 2 to 12% by weight, wherein the specified amounts relate to the total weight of the cleaning or nourishing composition.

The group of particularly preferred anionic surfactants includes, for example:
ether carboxylic acids of formula R—O—(CH₂—CH₂O)ₓ—CH₂—COOH, in which R is a linear or branched, saturated or unsaturated alkyl group having 8 to 30 C atoms and x=0 or 1 to 16,
acyl glutamate and/or (acyl)isethionates having 8 to 24 C atoms in the acyl group,
sulfosuccinic acid mono and/or dialkyl esters having 8 to 24 C atoms in the alkyl group and 1 to 6 oxyethyl groups, and/or
alkyl sulfate and/or alkylpolyglycolether sulfate salts of formula R—(OCH₂—CH₂)ₓ—OSO₃—⁻X⁺, in which R is preferably a linear or branched, saturated or unsaturated alkyl group having 8 to 30 C atoms, x means the number 0 or 1 to 12, and X means an alkali or alkaline earth, ammonium, or alkanolamine ion.

Particularly preferred anionic surfactants are straight-chain or branched alkyl ether sulfates of the aforementioned formula which include an alkyl group having 8 to 18, in particular having 10 to 16 C atoms, and also 1 to 6 and in particular 2 to 4 ethylene oxide units. The sodium, magnesium and/or triethanolamine salts of linear or branched lauryl, tridecyl, and/or myristyl sulfates having a degree of ethoxylation of from 2 to 4 are particularly preferred.

In a further preferred embodiment of the second subject of the invention, the cleaning and nourishing composition according to the invention is formulated as a conditioner and preferably includes at least one cationic surfactant in a preferred proportion by weight of preferably from 0.01 to 5% by weight, more preferably from 0.05 to 2% by weight, and in particular from 0.1 to 0.5% by weight, wherein the specified amounts relate to the total weight of the cleaning or nourishing composition.

Suitable cationic surfactants are, for example, quaternary ammonium compounds, esterquats and/or amidoamines.

Preferred quaternary ammonium compounds are ammonium halides, in particular chlorides and bromides, such as alkyltrimethylammonium chlorides, dialkyldimethylammonium chlorides, and trialkylmethylammonium chlorides, cetyl trimethyl ammonium chloride, stearyl trimethyl ammonium chloride, distearyl dimethyl ammonium chloride, lauryl dimethyl ammonium chloride, lauryl dimethyl benzyl ammonium chloride, and tricetyl methyl ammonium chloride and also the imidazolium compounds known by the INCI names Quaternium-27, Quaternium-83 and Quaternium-87. The alkyl chains of the above-mentioned surfactants preferably have 10 to 18 carbon atoms.

Esterquats are substances which include both an ester function and at least one quaternary ammonium group as structural element. Preferred esterquats are quaternized ester salts of fatty acids with triethanolamine, quaternized ester salts of fatty acids with diethanol alkyl amines, and quaternized ester salts of fatty acids with 1,2-dihydroxypropyldialkyl amines. Such products are sold for example under the trade name Stepantex®, Dehyquart®, Armocare®, and Quartamin®.

Alkylamidoamines are usually produced by amidation of natural or synthetic fatty acids and fatty acid cuts with dialkylaminoamines. A particularly suitable compound from this substance group is the stearamidopropyl dimethylamine commercially available under the name Tegoamid® S 18.

With regard to preferred divalent salts of monobasic organic acids or with regard to preferred mixtures of monobasic organic acids (b1) and divalent inorganic salts (b2), reference is made to the method forming the first subject of the invention.

In a third preferred embodiment of the second subject of the invention, the cosmetic cleaning and/or nourishing composition includes—in relation to its weight—
preferably 0.04 to 0.40 mol/kg, preferably 0.06 to 0.30 mol/kg, and in particular 0.07 to 0.20 mol/kg of the salt a), or

preferably 0.02 to 0.40 mol/kg, preferably 0.03 to 0.30 mol/kg, and in particular 0.05 to 0.20 mol/kg of the monobasic organic acid (b1), and preferably 0.05 to 0.40 mol/kg, more preferably 0.06 to 0.30 mol/kg, and in particular 0.07 to 0.20 mol/kg of the divalent inorganic salt (b2).

It has been found that the efficacy of the cleaning and/or nourishing composition can be increased yet further still if a specific hair-conditioning active substance is added to it.

In particular, the color brilliance of the artificially produced hair color could be stabilized and maintained as a result.

In a fourth preferred embodiment, the cleaning and/or nourishing composition forming the second subject of the invention additionally includes at least one hair-conditioning active substance in a proportion by weight of from 0.01 to 10% by weight in the total weight of the cosmetic cleaning or nourishing composition.

Suitable hair-conditioning active substances are preferably understood to be cationic nourishing polymers, natural, mineral or synthetic oil, fat or wax components, vitamins and/or protein hydrolysates.

Suitable protein hydrolysates are understood to be product mixtures which can be obtained by acid-catalysed, base-catalysed or enzymatically catalysed degradation of proteins.

Protein hydrolysates of plant, animal and/or marine origin can be used.

Animal protein hydrolysates are, for example, elastin, collagen, keratin, silk and milk protein hydrolysates, which can also be present in the form of salts. Such products are marketed for example under the trade names Dehylan® (Cognis), Promois® (Interorgana) Collapuron® (Cognis), Nutrilan® (Cognis), Gelita-Sol® (German gelatin factories Stoess & Co), Lexein® (Inolex) and Kerasol® (Croda).

Protein hydrolysates of plant origin are preferred, for example soy, almond, rice, pea, potato and wheat protein hydrolysates. Such products are obtainable, for example, under the trade names Gluadin® (Cognis), DiaMin® (Diamalt), Lexein® (Inolex) and Crotein® (Croda).

In addition, cationized protein hydrolysates can be used, wherein the basic protein hydrolysate can originate from animals, for example from collagen, milk or keratin, from plants, for example, from wheat, maize, rice, potatoes, soya or almonds, from marine life, for example, from fish collagen or algae, or from biotechnologically obtained protein hydrolysates. The protein hydrolysates forming the basis of the cationic derivatives can be obtained from the corresponding proteins by a chemical hydrolysis, particularly alkaline or acid hydrolysis, by an enzymatic hydrolysis and/or a combination of both types of hydrolysis. The hydrolysis of proteins generally produces a protein hydrolysate with a molecular weight distribution from about 100 daltons up to several thousand daltons. Cationic protein hydrolysates that are preferred are those of which the base protein content has a molecular weight of 100 to 25,000 daltons, preferably 250 to 5,000 daltons. Moreover, cationic protein hydrolysates are understood to include quaternized amino acids and their mixtures. Quaternization of the protein hydrolysates or the amino acids is often carried out using quaternary ammonium salts such as, for example, N,N-dimethyl-N-(n-alkyl)-N-(2-hydroxy-3-chloro-n-propyl) ammonium halides. Moreover, the cationic protein hydrolysates can also be further derivatized. Typical examples of cationic protein hydrolysates and derivatives are the commercially available products known under the INCI names Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Lauryldimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein.

The protein hydrolysate(s) can be used in the cleaning or nourishing composition forming the second subject of the invention—in relation the total weight thereof—preferably in amounts of from 0.01 to 5% by weight, more preferably from 0.025 to 3% by weight, and in particular from 0.05 to 2% by weight.

Suitable cationic polymers are, for example:
quaternized cellulose derivatives, as are commercially available for example under the names Celquat® and Polymer JR®,
hydrophobically modified cellulose derivatives, for example the cationic polymers sold under the trade name SoftCat®,
cationic alkyl polyglycosides,
cationized honey, for example the commercial product Honeyquat® 50,
cationic guar derivatives, such as in particular the products sold under the trade names Cosmedia®Guar N-Hance® and Jaguar®,
polymeric dimethyl diallyl ammonium salts and copolymers thereof with esters and amides of acrylic acid and methacrylic acid. The products commercially available under the names Merquat® 100 (poly(dimethyl diallyl ammonium chloride)) and Merquat® 550 (dimethyl diallyl ammonium chloride-acrylamide copolymer) are examples of such cationic polymers,
copolymers of vinylpyrrolidone with quaternized derivatives of dialkylamino alkyl acrylate and methacrylate, such as vinylpyrrolidone-dimethylaminoethyl methacrylate copolymers quaternized with diethyl sulfate. Such compounds are commercially obtainable under the names Gafquat® 734 and Gafquat® 755,
vinylpyrrolidone-vinylimidazolium methochloride copolymers, as are sold under the names Luviquat® FC 370, FC 550, FC 905 and HM 552,
quaternized polyvinyl alcohol,
   and the polymers known under the names
Polyquaternium 2, Polyquaternium 17, Polyquaternium 18, Polyquaternium-24, Polyquaternium 27, Polyquaternium-32, Polyquaternium-37, Polyquaternium 74 and Polyquaternium 89.

Preferred cationic polymers are cationic polysaccharide polymers such as quaternized cellulose polymers, hydrophobically modified cationic cellulose derivatives and/or cationic guar derivatives which are particularly preferably selected from the polymers known under the INCI names Guar Hydroxypropyltrimonium Chloride, Polyquaternium-10 and/or Polyquaternium-67.

The cationic polymer(s) can be used in the cleaning or nourishing composition forming the second subject of the invention—in relation to the total weight thereof—preferably in an amount of from 0.01 to 10% by weight, more preferably 0.05 to 5% by weight, and in particular 0.1 to 3% by weight.

Vitamins are preferably understood to mean the following vitamins, provitamins and vitamin precursors and derivatives thereof:
Vitamin A: the group of substances referred to as vitamin A includes retinol (Vitamin A₁) and 3,4-didehydroretinol (Vitamin A₂). β-carotin is the provitamin of retinol. Examples of suitable vitamin A components include vitamin A acid and esters thereof, vitamin A aldehyde, and vitamin A alcohol as well as esters thereof, such as the palmitate and acetate.
Vitamin B: the vitamin B group or the vitamin B complex includes, inter alia,
   Vitamin B₁ (thiamin)
   Vitamin B₂ (riboflavin)
   Vitamin B₃. This often includes the compounds of nicotinic acid and nicotinic acid amide (niacinamide).
   Vitamin B₅ (pantothenic acid and panthenol). In the context of this group, panthenol is preferably used. Usable derivatives of panthenol are especially the esters and ethers of panthenol, pantolactone, and also cationically derivatized panthenols. Specific representatives are, for example, panthenol triacetate, panthenol monoethyl ether and monoacetate thereof, as well as cationic panthenol derivatives.
   Vitamin B₆ (pyridoxine and also pyridoxamine and pyridoxal).
Vitamin C (ascorbic acid): the use in the form of the palmitic acid ester, the glucosides, or phosphates can be preferred. The use in combination with tocopherols can also be preferred.
Vitamin E (tocopherols, in particular α-tocopherol).
Vitamin F: the term “vitamin F” is usually understood to mean essential fatty acids, in particular linoleic acid, linolenic acid and arachidonic acid.
Vitamin H: The compound (3aS,4S,6aR)-2-oxohexahydrothienol[3,4-d]-imidazol-4-valeric acid denotes vitamin H, for which the trivial name however (biotin) has become accepted.

The cleaning or nourishing composition according to the invention forming the second subject of the invention can preferably include vitamins, provitamins and vitamin precursors from groups A, B, E and H.

Nicotinic acid amide, biotin, pantolactone and/or panthenol are preferred in particular.

Vitamins, vitamin derivatives and/or vitamin precursors can be used in the cleaning or nourishing composition according to the invention forming the second subject of the invention—in relation to the total weight thereof—preferably in an amount of from 0.001 to 2% by weight, particularly preferably from 0.005 to 1% by weight, and in particular from 0.01 to 0.5% by weight.

Suitable natural (plant) oils are usually understood to be triglycerides and mixtures of triglycerides. Preferred natural oils are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheatgerm oil, pumpkin seed oil, mallow oil, hazelnut oil, safflower oil, canola oil, sasanqua oil, jojoba oil, rambutan oil, cocoa butter and/or Shea butter.

In particular, petroleum oils, paraffin oils and isoparaffin oils as well as synthetic hydrocarbons are used as mineral oils. An example of a hydrocarbon that can be used is, for example, the commercially available 1,3-di-(2-ethylhexyl)-cyclohexane (Cetiol® S).

A dialkyl ether can also be used as oil component.

Dialkyl ethers that can be used are in particular di-n-alkyl ethers having a total of between 12 and 36 C atoms, in particular 12 to 24 C atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether and also di-tert-butyl ether, di-iso-pentyl ether, di-3-ethyl decyl ether, tert-butyl-n-octyl ether, iso-pentyl-n-octyl ether and 2-methylpentyl-n-octyl ether.

The di-n-octyl ether commercially available under the name Cetiol® OE is particularly preferred.

Silicone compounds are considered with preference as synthetic oils.

Silicones provide excellent conditioning properties when applied to hair. In particular, they result in an improved combinability of the hair in the wet and dry states and in many cases have a positive effect on the manageability of the hair and the softness of the hair.

It is therefore desirable to use silicones in cosmetic hair treatment agents. Suitable silicones can be selected from:
a) polyalkyl siloxanes, polyaryl siloxanes, polyalkylaryl siloxanes, which are volatile or not volatile, straight-chained, branched or cyclic, cross-linked or non-cross-linked;
b) polysiloxanes which in their general structure include one or more organofunctional groups selected from
   i. substituted or unsubstituted aminated groups;
   ii. (per)fluorinated groups;
   iii. thiol groups;
   iv. carboxylate groups;
   v. hydroxylated groups;
   vi. alkoxylated groups;
   vii. acyloxyalkyl groups;
   viii. amphoteric groups;
   ix. bisulfate groups;
   x. hydroxyacylamino groups;
   xi. carboxy groups;
   xii. sulfonic acid groups; and
   xiii. sulfate or thiosulfate groups;
c) linear polysiloxane (A)-polyoxyalkylene (B) block copolymers of the type (A-B)ₙ with n>3;
d) grafted silicone polymers with non-silicone-containing, organic parent structure which consist of an organic main chain formed from organic monomers including no silicone, onto which at least one polysiloxane macromer has been grafted in the chain and also optionally at least at one chain end;
e) grafted silicone polymers with polysiloxane parent structure, onto which non-silicone-containing organic monomers have been grafted, which comprise a polysiloxane main chain onto which at least one organic macromer including no silicone has been grafted in the chain and optionally at least at one of the ends thereof.
f) or mixtures thereof.

Fatty substances are understood to mean fatty acids, fatty alcohols and also natural and synthetic waxes, which can be present either in solid form or in liquid form in aqueous dispersion.

Linear and/or branched, saturated and/or unsaturated fatty acids having 6-30 carbon atoms can be used as fatty acids. Fatty acids having 10-22 carbon atoms are preferred. Examples include the isostearic acids, such as the commercial products Emersol® 871 and Emersol® 875, and isopalmitic acids such as the commercial product Edenor® IP 95, and also all further fatty acids sold under the commercial names Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof.

The fatty acid cuts obtainable from coconut oil or palm oil are usually particularly preferred; the use of stearic acid is generally preferred in particular.

Saturated, mono- or polyunsaturated, branched or unbranched fatty alcohols having C₆-C_{30,} preferably C₁₀-C_{22,} and very particularly preferably C₁₂-C₂₂ carbon atoms can be used as fatty alcohols. For example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, caprin alcohol, linoleyl alcohol, linolenyl alcohol and behenyl alcohol, and the guerbet alcohols thereof can be used, where this list is intended to be exemplary and non-limiting. The fatty alcohols, however, preferably originate from natural fatty acids, wherein recovery from the esters of fatty acids by reduction can usually be assumed. Fatty alcohol cuts which are produced by reduction of naturally occurring triglycerides, such as beef tallow, palm oil, peanut oil, rapeseed oil, cottonseed oil, soybean oil, sunflower oil and linseed oil, or fatty acid esters formed from the transesterification products thereof with corresponding alcohols and which therefore represent a mixture of different fatty alcohols can also be used in accordance with the invention. Such substances can be purchased for example under the names Stenol®, for example Stenol® 1618, or Lanette®, for example Lanette® O, or Lorol®, for example Lorol® C8, Lorol® C14, Lorol® C18, Lorol® C8-18, HD-Ocenol®, Crodacol®, for example Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, lanolin alcohols can also be used in accordance with the invention, as can be purchased for example under the names Corona®, White Swan®, Coronet® or Fluilan®.

Solid paraffins or isoparaffins, carnauba wax, beeswax, candelilla wax, ozokerite, ceresin, spermaceti, sunflower wax, fruit wax, such as apple wax or citrus wax, and microwax from PE or PP can be used as natural or synthetic waxes. Such waxes are obtainable for example from the company Kahl & Co., Trittau.

Further fatty substances are, for example
ester oils. Ester oils are understood to mean the esters of C₆-C₃₀ fatty acids having C₂-C₃₀ fatty alcohols. The monoesters of fatty acids with alcohols having 2 to 24 C atoms are preferred. Examples of used fatty acid components in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof.
Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachyl alcohol, gadoleyl alcohol, behenyl alcohol, erucyl alcohol and brassidyl alcohol and technical mixtures thereof. Particularly preferred are isopropyl myristate (Rilanit® IPM), isononanoic acid C16-C18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® LC), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).
Dicarboxylic acid esters such as di-n-butyl adipate, di-(2-ethylhexyl) adipate, di-(2-ethylhexyl) succinate and di-isotridecyl acelaat, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate, and neopentyl glycol dicaprylate,
symmetrical, asymmetrical or cyclic esters of carbonic acid with fatty alcohols, glycerol carbonate or dicaprylyl carbonate (Cetiol® CC),
ethoxylated or non-ethoxylated mono-, di-, and tri-fatty acid esters of saturated and/or unsaturated linear and/or branched fatty acids with glycerol, such as Monomuls® 90-O18, Monomuls® 90-L12, Cetiol® HE or Cutina® MD.

The proportion by weight of the oil, wax and/or fatty components in the total weight of the cleaning or nourishing composition according to the invention forming the second subject of the invention is preferably 0.01 to 10% by weight, particularly preferably 0.025 to 7.5% by weight, and in particular 0.05 to 5% by weight.

It has been found that the color brilliance of the hair can be particularly well stabilized with the cleaning or nourishing compositions according to the invention forming the second subject of the invention if the cleaning or nourishing compositions include, as hair-conditioning active substance, at least one cationic polymer, preferably a cationic polysaccharide, and/or at least one plant oil and/or a silicone. The haptic properties such as the feel and smoothness of the dyed hair could additionally be improved by addition of these specific nourishing substances.

In a further preferred embodiment, the cleaning and/or nourishing compositions forming the second subject of the invention therefore additionally include, as hair-conditioning active substance, a cationic polymer—in particular a cationic polymer—in particular a cationic polysaccharide—and/or a plant oil and/or a silicone.

Chelating and/or complexing agents, in particular nitrogen-containing complexing and/or chelating agents, can have a negative effect on the salts obligatorily included in the cleaning or nourishing compositions by complexing the cations of the partially to fully dissolved salts and thus interfering with the efficacy thereof.

In a further preferred embodiment, the cleaning or nourishing compositions forming the second subject of the invention are therefore substantially free from nitrogen-containing complexing and/or chelating agents.

In a fifth preferred embodiment, cleaning or nourishing compositions according to the invention forming the second subject of the invention are characterized in that they include—in relation to their weight—
a) preferably 0.04 to 0.40 mol/kg, more preferably 0.06 to 0.30 mol/kg, and in particular 0.07 to 0.20 mol/kg of calcium lactate, or
   preferably 0.02 to 0.40 mol/kg, more preferably 0.03 to 0.30 mol/kg, and in particular 0.05 to 0.20 mol/kg of lactic acid and preferably 0.05 to 0.40 mol/kg, more preferably 0.06 to 0.30 mol/kg, and in particular 0.07 to 0.20 mol/kg of calcium chloride or calcium hydroxide,
b) preferably 0.5 to 20% by weight, more preferably from 1 to 15, and in particular from 2 to 12% by weight of at least one anionic surfactant and/or preferably 0.01 to 5% by weight, more preferably from 0.05 to 2% by weight, and in particular from 0.1 to 0.5% by weight of at least one cationic surfactant, and
c) preferably 0.01 to 10% by weight, more preferably 0.05 to 5% by weight, and in particular 0.1 to 3% by weight of at least one cationic polysaccharide, and have a pH value ranging from 4.2 to 4.7, preferably from 4.25 to 4.65, and in particular from 4.3 to 4.6.

Within this embodiment, cleaning or nourishing compositions forming the second subject of the invention that are particularly preferred are those which contain
a) preferably 0.04 to 0.40 mol/kg, more preferably 0.06 to 0.30 mol/kg, and in particular 0.07 to 0.20 mol/kg of calcium lactate, or
   preferably 0.02 to 0.40 mol/kg, more preferably 0.03 to 0.30 mol/kg, and in particular 0.05 to 0.20 mol/kg of lactic acid and preferably 0.05 to 0.40 mol/kg, more preferably 0.06 to 0.30 mol/kg, and in particular 0.07 to 0.20 mol/kg of calcium chloride or calcium hydroxide,
b) preferably 0.5 to 20% by weight of at least one alkyl sulfate and/or alkylpolyglycolether sulfate salt of formula R—(OCH₂—CH₂)ₓ—OSO₃—⁻X⁺, in which R is preferably a linear or branched, saturated or unsaturated alkyl group having 8 to 30 C atoms, x means the number 0 or 1 to 12, and X means an alkali or alkaline earth, ammonium, or alkanolamine ion, and
c) preferably 0.01 to 10% by weight of at least one of the cationic polysaccharides known under the INCI names Polyquaternium-10, Guar Hydroxypropyltrimonium Chloride and/or Polyquaternium-67, and
   have a pH value ranging from 4.3 to 4.6.

Within this embodiment, cleaning or nourishing compositions forming the second subject of the invention that are particularly preferred are also those that include
a) preferably 0.04 to 0.40 mol/kg, more preferably 0.06 to 0.30 mol/kg, and in particular 0.07 to 0.20 mol/kg of calcium lactate, or
   preferably 0.02 to 0.40 mol/kg, more preferably 0.03 to 0.30 mol/kg, and in particular 0.05 to 0.20 mol/kg of lactic acid and preferably 0.05 to 0.40 mol/kg, more preferably 0.06 to 0.30 mol/kg, and in particular 0.07 to 0.20 mol/kg of calcium chloride or calcium hydroxide,
b) preferably 0.01 to 5% by weight of cetyl trimethyl ammonium chloride, stearyl trimethyl ammonium chloride, behenyl trimethyl ammonium chloride, distearyl dimethyl ammonium chloride, lauryl dimethyl ammonium chloride, lauryl dimethyl benzyl ammonium chloride and/or tricetyl methyl ammonium chloride, and
c) preferably 0.01 to 10% by weight of at least one of the cationic polysaccharides known under the INCI names Polyquaternium-10, Guar Hydroxypropyltrimonium Chloride and/or Polyquaternium-67, and have a pH value ranging from 4.3 to 4.6.

In a sixth preferred embodiment, cleaning or nourishing compositions according to the invention forming the second subject of the invention are characterized in that they include—in relation to their weight—
a) preferably 0.04 to 0.40 mol/kg, more preferably 0.06 to 0.30 mol/kg, and in particular 0.07 to 0.20 mol/kg of calcium lactate, or
   preferably 0.02 to 0.40 mol/kg, more preferably 0.03 to 0.30 mol/kg, and in particular 0.05 to 0.20 mol/kg of lactic acid and preferably 0.05 to 0.40 mol/kg, more preferably 0.06 to 0.30 mol/kg, and in particular 0.07 to 0.20 mol/kg of calcium chloride or calcium hydroxide,
b) preferably 0.5 to 20% by weight, more preferably from 1 to 15, and in particular from 2 to 12% by weight of at least one anionic surfactant and/or preferably 0.01 to 5% by weight, more preferably from 0.05 to 2% by weight, and in particular from 0.1 to 0.5% by weight of at least one cationic surfactant, and
c) preferably 0.01 to 10% by weight, particularly preferably 0.025 to 7.5% by weight, and in particular 0.05 to 5% by weight of at least one natural, mineral or synthetic oil, fat or wax component, and
   have a pH value ranging from 4.2 to 4.7, preferably from 4.25 to 4.65, and in particular from 4.3 to 4.6.

Within this embodiment, cleaning or nourishing compositions forming the second subject of the invention that are particularly preferred are those which include
a) preferably 0.04 to 0.40 mol/kg, more preferably 0.06 to 0.30 mol/kg, and in particular 0.07 to 0.20 mol/kg of calcium lactate, or
   preferably 0.02 to 0.40 mol/kg, more preferably 0.03 to 0.30 mol/kg, and in particular 0.05 to 0.20 mol/kg of lactic acid and preferably 0.05 to 0.40 mol/kg, more preferably 0.06 to 0.30 mol/kg, and in particular 0.07 to 0.20 mol/kg of calcium chloride or calcium hydroxide,
b) preferably 0.5 to 20% by weight of at least one alkyl sulfate and/or alkylpolyglycolether sulfate salt of formula R—(OCH₂—CH₂)ₓ—OSO₃—⁻X⁺, in which R is preferably a linear or branched, saturated or unsaturated alkyl group having 8 to 30 C atoms, x means the number 0 or 1 to 12, and X means an alkali or alkaline earth, ammonium, or alkanolamine ion, and
c) preferably 0.01 to 10% by weight of at least one plant oil and/or a silicone, and have a pH value ranging from 4.3 to 4.6.

Within this embodiment, cleaning or nourishing compositions forming the second subject of the invention that are particularly preferred are also those that include
a) preferably 0.04 to 0.40 mol/kg, more preferably 0.06 to 0.30 mol/kg, and in particular 0.07 to 0.20 mol/kg of calcium lactate, or
   preferably 0.02 to 0.40 mol/kg, more preferably 0.03 to 0.30 mol/kg, and in particular 0.05 to 0.20 mol/kg of lactic acid and preferably 0.05 to 0.40 mol/kg, more preferably 0.06 to 0.30 mol/kg, and in particular 0.07 to 0.20 mol/kg of calcium chloride or calcium hydroxide,
b) preferably 0.01 to 5% by weight of cetyl trimethyl ammonium chloride, stearyl trimethyl ammonium chloride, behenyl trimethyl ammonium chloride, distearyl dimethyl ammonium chloride, lauryl dimethyl ammonium chloride, lauryl dimethyl benzyl ammonium chloride and/or tricetyl methyl ammonium chloride, and
c) preferably 0.01 to 10% by weight of at least one plant oil and/or a silicone, and have a pH value ranging from 4.3 to 4.6.

The cleaning or nourishing compositions forming the second subject of the invention can comprise further preferred ingredients which provide the compositions with their advantageous properties, said further ingredients including
non-ionic and/or amphoteric surfactants,
anti-dandruff active substances,
pearlescent agents and/or opacifiers.

The group of suitable non-ionic surfactants includes, for example:
C₈-C₃₀ fatty acid mono- and diesters of addition products of 1 to 30 mol Ethylenoxidan Glycerol,
amine oxides,
addition products of 2 to 50 mol ethylene oxide and/or 0 to 5 mol propylene oxide with linear and branched fatty alcohols having 8 to 30 C atoms, with fatty acids having 8 to 30 C atoms, and with alkyl phenols having 8 to 15 C atoms in the alkyl group,
sorbitol fatty acid esters and addition products of ethylene oxide with sorbitol fatty acid esters, such as polysorbates,
fatty acid alkanolamides,
sugar fatty acid esters and addition products of ethylene oxide with sugar fatty acid esters,
addition products of ethylene oxide with fatty acid alkanolamides and fatty amines and/or
alkyl polyglucosides of general formula RO-[G]ₓ, in which R stands for an alkyl and/or alkenyl group having 4 to 22 C atoms, G stands for a sugar residue having 5 or 6 C atoms, and x stands for numbers from 1 to 10.

Particularly suitable alkyl polyglucosides can be derived from aldoses and/or ketoses having 5 or 6 carbon atoms, preferably from glucose. The group R particularly preferably stands for an alkyl group having 6 to 20 and in particular having 8 to 18 carbon atoms. The index number x in the general formula RO-[G][x] stands for the degree of oligomerisation (DP), i.e. for the distribution of the mono- and oligoglycosides. The index number x preferably has a value ranging from 1 to 6, particularly preferably ranging from 1 to 3, wherein it does not have to be an integer, but can be a fractional number, which can be analytically determined. Particularly preferred alkyl polyglucosides have a degree of oligomerisation between 1.2 and 1.5. Particularly suitable alkyl polyglucosides are known and are commercially available from various providers under the INCI names Decyl Glucoside, Lauryl Glucoside and Coco Glucoside.

Particularly suitable non-ionic surfactants/emulsifiers are alkyl polyglucosides, in particular alkyl polyglucosides based on hardened C₁₀₋₁₄ coconut alcohol having a DP of 1-3, as are commercially obtainable for example under the INCi name “Coco-Glucoside”.

The non-ionic surfactant(s) is/are used in the cleaning or nourishing compositions forming the second subject of the invention—in relation to the total weight thereof—preferably in an amount of from 1 to 15% by weight, more preferably from 1.5 to 14% by weight, particularly preferably from 2 to 12.5% by weight, and in particular from 2.5 to 10% by weight.

The group of suitable amphoteric and/or zwitterionic surfactants include what are known as the betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines including in each case 8 to 18 carbon atoms in the alkyl or acyl group and cocoacylaminoethyl hydroxyethyl carboxymethyl glycinate.

Amphoteric surfactants are understood to be surface-active compounds which are capable of forming inner salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkyl aminopropionic acids, and alkyl amino acetic acids each having 8 to 24 C atoms in the alkyl group. Typical examples of amphoteric and zwitterionic surfactants are alkylbetaines, alkylamidobetaines, aminopropionates, aminoglycinates, imidazolinium betaines, and sulfobetaines.

Particularly preferred amphoteric surfactants are N-coco-alkylaminopropionate, coco-acylaminoethylaminopropionate, the fatty acid amide derivative known under the INCI name Cocamidopropyl Betaine, and C₁₂-C₁₈ acyl sarcosine.

The total amount of amphoteric and/or zwitterionic surfactants in the cleaning or nourishing compositions forming the second subject of the invention is preferably 1 to 6% by weight, more preferably from 1.2 to 5% by weight, particularly preferably 1.4 to 4.5% by weight, and in particular 1.5 to 4% by weight, wherein the specified amounts relate to the total weight of the cosmetic compositions.

Anti-dandruff active substances can be used in the cleaning or nourishing compositions forming the second subject of the invention—in relation to the total weight thereof—preferably in amounts of from 0.01 to 10% by weight, more preferably from 0.025 to 7.5% by weight, particularly preferably from 0.05 to 5% by weight, and in particular from 0.075 to 3% by weight.

Suitable anti-dandruff active substances can be selected from piroctone olamine, climbazole, zinc pyrithione, ketoconazoles, salicylic acid, sulfur, selenium sulfide, tar preparations, undecenoic acid derivatives, burdock extracts, poplar extracts, stinging nettle extracts, walnut shell extracts, birch extracts, willow bark extracts, rosemary extracts and/or arnica extracts. Climbazole, zinc pyrithione, piroctone olamine and/or salicylic acid are preferred.

Suitable opacifiers and/or pearlescent agents can be used in the cleaning or nourishing compositions forming the second subject of the invention—in relation to the total weight thereof—preferably in amounts of (in each case) 0.001 to 5% by weight, more preferably from 0.005 to 4% by weight, particularly preferably from 0.01 to 3% by weight, and in particular from 0.05 to 2% by weight.

Suitable pearlescent agents and opacifiers are to be understood, for example, to be
mono- and/or diesters of ethylene glycol, 1,2-propanediol and/or glycerol having C₈-C₂₄ fatty acids,
esters of polyethylene glycols having C₈-C₂₄ fatty acids,
TiO₂ or TiO₂-coated synthetic or natural mica and/or
styrene/acrylate copolymers.

The opacifiers and/or pearlescent agents known under the following INCI names are particularly suitable:
Glycol Distearate, such as the commercial product Cutina® AGS from Cognis, Glycol Monostearate, such as the commercial product Cutina® RGMS from Cognis, PEG-3 Distearate, such as the commercial product Genapol® TS from Clariant, PEG-2 Distearate, such as the commercial product Kessco® DEGMS from Akzo Nobel, Propylene Glycol Stearate, such as the commercial product Tegin® P from Goldschmidt and/or Styrene/Acrylates Copolymers, such as the commercial products Joncryl® 67 from Johnson Polymers, Suprawal® WS from BASF and/or Acusol® OP 301 from Rohm & Haas.

Further active substances, auxiliaries and additives which can be included in the cleaning or nourishing compositions according to the invention forming the second subject of the invention are, for example:
plant extracts,
humectants,
perfumes,
UV filters,
thickening agents such as gelatins or plant gums, for example agar-agar, guar gum, alginates, xanthan gum, gum arabic, karaya gum, carob seed meal, linseed gums, dextrans, cellulose derivatives, for example methyl cellulose, hydroxyalkyl cellulose and carboxymethyl cellulose, starch fractions and derivatives such as amylose, amylopectin and dextrins, clays and phyllosilicates such as bentonite or fully synthetic hydrocolloids such as polyvinyl alcohol, the Ca, Mg or Zn soaps,
structuring agents such as maleic acid and lactic acid,
dimethyl isosorbide,
cyclodextrins,
active substances to improve the fibre structure, in particular mono-, di- and oligosaccharides such as glucose, galactose, fructose, fruit sugar and lactose,
dyes to color the agent,
active substances such as bisabolol,
ceramides. Ceramides are understood to be N-acyl sphingosine (fatty acid amides of sphingosine) or synthetic analogues of such lipids (known as pseudoceramides),
propellants such as propane-butane mixtures, N20, dimethyl ether, CO2 and air,
antioxidants,
preservatives, such as sodium benzoate or salicylic acid,
viscosity adjusters such as salts (NaCl).

A third subject of the invention is the use of the cleaning or nourishing compositions according to the invention for reducing and/or preventing the bleaching and/or fading of artificially produced hair colors and for improving the color intensity and/or the color fidelity.

### EXAMPLES

1) Practical examples: The following cleaning and nourishing compositions according to the invention were prepared (the specified amounts are in % by weight):

| | Shampoo 1 | Shampoo 2 |
|---|---|---|
| Sodium Laureth Sulfate | 11.00 | 11.00 |
| Cocamidopropyl Betaine | 1.00 | 1.50 |
| Disodium | 0.50 | |
| Cocoamphodiacetate | | |
| Cocamide MEA | 0.50 | 0.50 |
| PEG-12 Dimethicone | 0.50 | 0.30 |
| Glycol Distearate | 1.20 | |
| PEG-7 Glyceryl Cocoate | 0.40 | 0.60 |
| Polyquaternium-10 | 0.90 | 0.60 |
| Panthenol | 0.30 | 0.20 |
| Apricot Kernel Oil | 0.05 | |
| Hydrogenated Castor Oil | 0.20 | 0.10 |
| Lactic Acid | 2.23 | |
| Calcium Hydroxide | 0.542 | |
| Calcium Lactate | | 1.50 |
| Preservatives, Perfume, | q.s. | q.s. |
| possibly Acidifer | | |
| Water | to 100 | to 100 |
| pH | 4.5 | |

### a) Color-Protect Shampoos

The above-mentioned shampoo 1 was applied to the wet hair in accordance with the method according to the invention and was rinsed out after being left to take effect for approximately one minute. The hair, after having been cleaned, had a brilliant hair color, which had not changed. In addition, the hair had a visually appealing shine and felt soft.

### b) Conditioners

| | Conditioner 1 | Conditioner 2 |
|---|---|---|
| Cetearyl Alcohol | 2.50 | 2.50 |
| Quaternium-87 | 2.00 | 2.00 |
| Lactic Acid | 1.60 | |
| Propylene Glycol | 0.50 | 0.50 |
| Isopropyl Myristate | 0.30 | 0.30 |
| Distearoylethyl Hydroxyethylmonium | 0.50 | 0.50 |
| Methosulfate | | |
| Calcium Hydroxide | 0.54 | |
| Phenoxyethanol | 0.40 | 0.40 |
| Stearamidopropyl Dimethylamine | 0.30 | 0.30 |
| Perfume | 0.25 | 0.25 |
| Sodium Methylparaben | 0.20 | 0.20 |
| Polyquaternium-37 | 0.20 | 0.20 |
| Dicaprylyl Carbonate | 0.20 | 0.20 |
| Panthenol | 0.10 | 0.10 |
| Benzophenone-4 | 0.05 | 0.05 |
| Amidodimethicone/Morpholinomethyl | 0.02 | 0.02 |
| Silsesquioxane Copolymer | | |
| Glycerol | 0.01 | 0.01 |
| Hydrolysed Keratin | 0.01 | 0.01 |
| Calcium Lactate | | 1.40 |
| Preservatives, Perfume, possibly | q.s. | q.s. |
| Acidifer | | |
| Water | to 100 | to 100 |
| pH | 4.3 | 4.3 |

2) Proof of efficacy: Strands of hair were tested which had been dyed using equal amounts of the same commercially available permanent hair dye compositions.

One set of hair strands was treated in accordance with the method according to the invention with a nourishing shampoo including 0.3 mol/kg of calcium lactate, whereas 5 further sets of hair strands were each washed in the same way with the same amount of commercially available color-protect shampoos including no calcium lactate. The treatment processes were repeated 5 times for each set of hair strands. The delta-E value (see below) was then determined for each set of hair strands. The results indicate that the hair color changed to the least extent following the treatment with nourishing shampoo 2.

The following shampoos were tested:

| Shampoos | pH value |
|---|---|
| 1. Nourishing shampoo without calcium lactate | 4.50 |
| 2. Nourishing shampoo with 0.3% by weight calcium lactate | 4.50 |
| 3. Color-protect shampoo, commercial product 1 | 5.70 |
| 4. Color-protect shampoo, commercial product 2 | 5.47 |
| 5. Color-protect shampoo, commercial product 3 | 4.32 |
| 6. Color-protect shampoo, commercial product 4 | 5.46 |

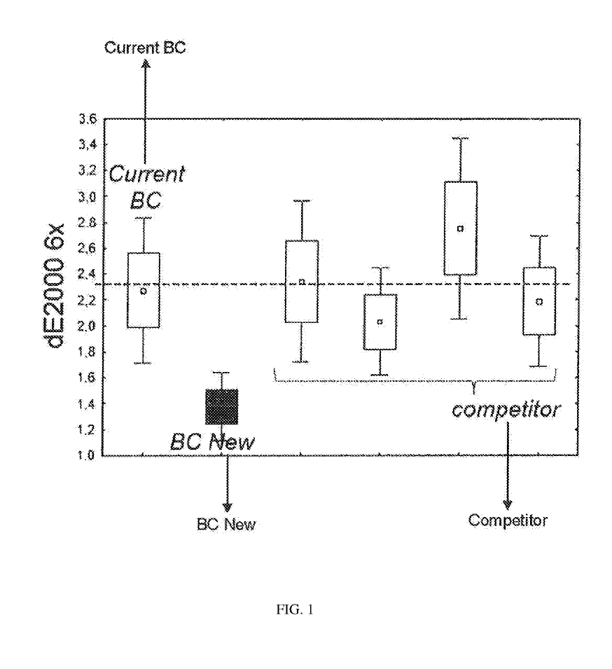

The attached FIGURE shows the delta-E values (see FIG. 1).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1-6. (canceled)

7. A cosmetic cleaning or nourishing composition for reducing and/or preventing the bleaching and/or fading of artificially produced hair colors, including—in relation to its weight—
(a)
i) 0.05 to 0.5 mol/kg of calcium lactate or
ii) 0.01 to 0.5 mol/kg of lactic acid (b1) and
0.05 to 0.5 mol/kg of (b2), wherein (b2) is selected from the group consisting of calcium chloride, calcium hydroxide and combinations thereof,
(b) at least one surfactant, selected from the group consisting of
anionic surfactants of the group consisting of alkyl(ether)sulfates, sulfosuccinates, ether carboxylic acids, acylglutamate and/or (acyl)isethionates, each with 8 to 24 C atoms in the acyl group, and mixtures of these surfactants, and
cationic surfactants selected from the group consisting of quaternary ammonium compounds, esterquats and amidoamines,
wherein the cleaning or nourishing composition has a pH value ranging from 4.2 to 4.7.

8. The cosmetic cleaning or nourishing composition according to claim 7, wherein the agent includes at least one anionic surfactant in a proportion of from 0.5 to 20% by weight, wherein the specified amounts relate to the total weight of the cleaning or nourishing composition.

9. The cosmetic cleaning or nourishing composition according to claim 7, wherein the agent includes at least one cationic surfactant in a proportion of from 0.01 to 5% by weight, wherein the specified amounts relate to the total weight of the cleaning or nourishing composition.

10. The cosmetic cleaning or nourishing composition according to claim 7, wherein the agent includes—in relation to its weight—
0.05 to 0.40 mol/kg of calcium lactate, or
0.02 to 0.40 mol/kg of (b1), and 0.05 to 0.40 mol/kg of (b2).

11. The cosmetic cleaning or nourishing composition according to claim 7, wherein the composition additionally includes at least one hair-conditioning active substance in a proportion of from 0.01 to 10% by weight in the total weight of the cosmetic cleaning or nourishing composition.

12. The cosmetic cleaning or nourishing composition according to claim 11, wherein the at least one hair-conditioning active substance is selected from cationic polymers selected from the group consisting of cationic polysaccharides, plant oils and silicones.

13. The cosmetic cleaning or nourishing composition according to claim 7, wherein the composition is substantially free from nitrogen-containing complexing and/or chelating agents.

14. The cosmetic cleaning or nourishing composition according to claim 7, including—in relation to its weight—
a) 0.05 to 0.40 mol/kg of calcium lactate, or 0.02 to 0.40 mol/kg of calcium chloride or calcium hydroxide, or
b) 0.5 to 20% by weight of at least one anionic surfactant and/or 0.01 to 5% by weight of at least one cationic surfactant, and
c) 0.01 to 10% by weight of at least one cationic polysaccharide, wherein the composition has a pH value ranging from 4.2 to 4.7.
